(19) United States
(12) Patent Application Publication
(10) Pub. No.: US 2025/0116604 A1
(43) Pub. Date: Apr. 10, 2025

(54) FLUORESCENCE DETECTION OF PERFLUOROALKYL SUBSTANCES USING LIVING CULTURES OF A NEWLY ISOLATED PSEUDOMONAS SP. BACTERIUM

(71) Applicant: The Government of the United States of America, as represented by the Secretary of the Navy, Arlington, VA (US)

(72) Inventors: Isabel Baker, Washington, DC (US); Lina Bird, Davidsonville, MD (US)

(73) Assignee: The Government of the United States of America, as represaented by the Secretary of the Navy, Arlington, VA (US)

(21) Appl. No.: 18/908,355

(22) Filed: Oct. 7, 2024

Related U.S. Application Data
(60) Provisional application No. 63/542,562, filed on Oct. 5, 2023.

(51) Int. Cl. G01N 21/64; C12N 1/20; C12Q 1/02; C12R 1/38; G01N 33/18

(52) U.S. Cl. CPC G01N 21/6428; C12N 1/205; C12Q 1/02; G01N 33/18; C12R 2001/38; G01N 2021/6439; G01N 2333/21

(57) ABSTRACT

A strain of Pseudomonas sp. bacterium, having been deposited with the Agricultural Research Culture Collection on 19 Jul. 2023 with a deposit number of NRRL B-68295, exhibits fluorescene in the presence of perfluoroalkyl molecules. The strain may be cultured and lyophilized. A method for detecting perfluoroalkyls includes contacting the strain with a sample and observing whether fluorescence occurs.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211498.

### CROSS-REFERENCE TO RELATED APPLICATIONS

This application claims priority to U.S. Provisional Patent Application No. 63/542,562, filed Oct. 5, 2023, which is incorporated by reference herein in its entirety.

### BACKGROUND

Perfluoroalkyl substances (PFAS) have been used in firefighting foams and industrial products (especially for waterproofing applications) for several decades. Only recently have the negative health and ecosystem effects of these molecules come to light. Their manufacture and use has led to their widespread presence in soil, groundwater, and drinking water. Efforts to regulate concentrations and remediate contamination by perfluoroalkyls are underway. Detection of these molecules in water samples is an ongoing challenge, due in part to its reliance on using liquid chromatography-mass spectrometry. While this technique provides maximal information regarding the composition of samples, it is costly, time-consuming, low-throughput, limited by access to proper instrumentation, and impractical for field-testing. Thus, a rapid and high throughput screening method for the presence of these molecules is of high interest.

Despite the pressing need for low-cost, easy-to-use, onsite-fieldable PFAS detection, a technique that meets these criteria has yet to arise. The currently available techniques for PFAS detection and quantification rely on expensive equipment (e.g., mass spectrometers) or materials (e.g., specialized molecularly imprinted polymers) that either cannot be readily brought into the field or require special infrastructure (e.g., facilities and/or expert labor) to be manufactured or maintained. In addition to these barriers to widespread use, most current methods are low-throughput or prohibitively slow.

It would be desirable to develop new systems and methods for detecting PFAS, preferably which are high-throughput, fast, and/or inexpensive relative to known systems and methods.

### BRIEF DESCRIPTION

In a first embodiment, a method of detecting a perfluoroalkyl material includes contacting a strain of Pseudomonas sp. bacterium, the strain having been deposited with the Agricultural Research Culture Collection on 19 Jul. 2023 with a deposit number of NRRL B-68295, with a sample to form a mixture; and observing the mixture to detect fluorescence thereof, wherein such fluorescence indicates the presence of the perfluoroalkyl material in the sample. The perfluoroalkyl material may include perfluorooctanoic acid (PFOA) and/or perfluorooctanesulfonic acid (PFOS). In some embodiments, the method further includes culturing the strain prior to contacting the strain with the sample. The strain may be provided in a liquid. In some embodiments, the strain was lyophilized prior to being provided to the liquid. The method can detect the fluorine-containing material in the sample at a concentration of 50 micrograms per liter. In some embodiments, the method further includes processing the sample prior to the contacting. Non-limiting examples of such processing include filtering the sample, concentrating the sample, and/or removing iron from the sample prior to the contacting. In some embodiments, the method further includes incubating the mixture for a predetermined time period prior to the observing. The predetermined time period may be in a range of about 12 hours to about 16 hours. In some embodiments, the incubation temperature is in a range of about 25° C. to about 35° C. The sample or mixture may be agitated prior to, during, or after the incubation. In some embodiments, the sample is a water (e.g., drinking water) sample. The method may be performed on a plurality of samples in parallel. In some embodiments, observing the mixture includes measuring fluorescence intensity of the mixture. The method may further include determining a concentration of the perfluoroalkyl material in the sample based on the fluorescence intensity.

A second embodiment is a cultured and lyophilized strain of Pseudomonas sp. bacterium, the strain having been deposited with the Agricultural Research Culture Collection on 19 Jul. 2023 with a deposit number of NRRL B-68295, wherein the strain, without having been modified, exhibits fluorescence when in the presence of perfluorooctanoic acid (PFOA) or perfluorooctanesulfonic acid (PFOS).

A third embodiment is a composition including the strain of the second embodiment.

These and other non-limiting aspects of the disclosure are more particularly set forth below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a flow chart illustrating a non-limiting example of a method for detecting perfluoroalkyl substances in accordance with some embodiments of the present disclosure.

FIG. 2 is a graph showing the fluorescent response of Pseudomonas sp. CBR-F to PFOA, excited at 425 nm and measured at 475 nm at 15.2 hours.

FIG. 3 is a graph showing the fluorescent response of Pseudomonas sp. CBR-F to PFOS, excited at 425 nm and measured at 475 nm at 15.2 hours.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of”, which allows the presence of only the named components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

Described herein is a new assay that overcomes limitations of known PFAS detection techniques. The assay is high-throughput (many samples can be measured at the same time), requires no specialized machinery (beyond a spectrophotometer, of which there are low cost, portable models), and can easily be performed without special training.

The assay involves a newly isolated bacterial species operable, without genetic or other modification, as a living sensor of the common perfluoroalkyl (PFAS) molecules perfluorooctanoic acid (PFOA) and perfluorooctanesulfonic acid (PFOS). These two molecules are common in contaminated environments and are the subject of EPA advisory limits.

The reported strain detects PFOA and PFOS at environmentally relevant concentrations and can be used with concentrated samples. The technique is also operable in the presence of organic contaminants and inorganic fluoride. These assays can be multiplexed, with 24 or even more samples tested in parallel. Furthermore, the assay uses low-cost and readily available materials in a format providing a rapid and portable assay, in contrast to current methods involving liquid chromatography and mass spectrometry.

In addition to PFOA and PFOS, it is believed that the systems and methods of the present application may be suitable for detecting other PFAS and potentially polyfluoroalkyl substances (e.g., polytetrafluoroethylene (PTFE)).

Non-limiting examples of additional PFAS include perfluorosulfonic acids (e.g., perfluorohexanesulfonic acid (PFHxS)), perfluoroalkyl carboxylic acids (e.g., perfluorononanoic acid (PFNA), perfluoroheptanoic acid (PFHpA), perfluorodecanoic acid (PFDA)), fluorotelomers, perfluorobutane sulfonamide (H-FBSA), perfluoropentanesulfonamide (PFPSA), perfluorohexanesulfonamide (PFHxSA), perfluoroheptanesulfonamide (PFHpSA), perfluorooctanesulfonamide (PFOSA), perfluorobutanesulfonyl fluoride (PFBSF), and perfluorooctanesulfonyl fluoride (PFOSF).

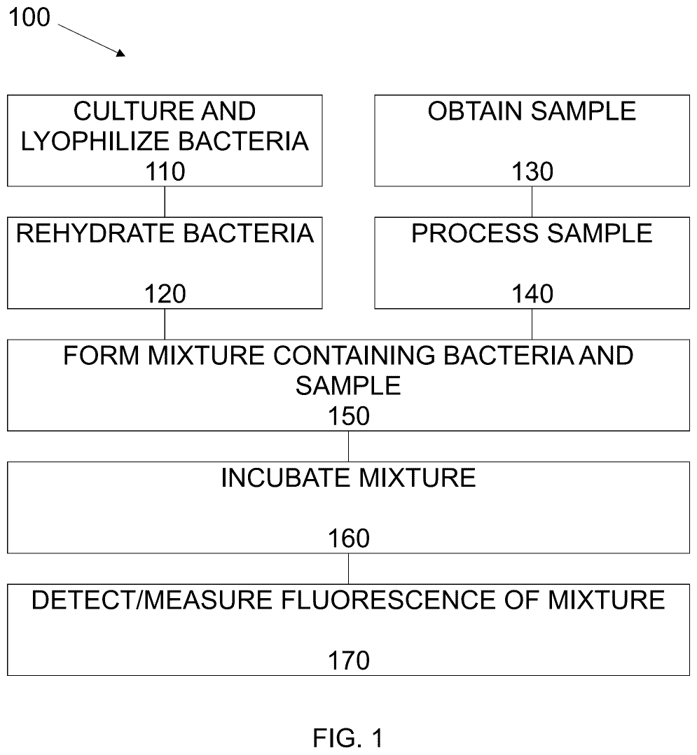

FIG. 1 is a flow chart illustrating a method 100 for detecting PFAS in accordance with some embodiments of the present disclosure. The method includes isolating the newly isolated bacterial species 110 and rehydrating the bacteria 120; and obtaining a sample 130 and processing the sample 140. The sample and bacteria are mixed to form a mixture 150. The mixture is incubated 160. Fluorescence is detected and/or measured to ascertain the presence and/or concentration of PFAS in the sample 170. It should be understood that not all of these steps are required. For example, lyophilization may not be required (e.g., if the strain is intended for use shortly after culture). If lyophilization does not occur, there would be no need for rehydration.

Techniques for culturing and lyophilizing bacteria 110 are known in the art. Bacterial culturing may be achieved by plating a small sample containing the bacteria on a solid agar medium and allowing the bacterial cells to grow into separate colonies, thereby enabling isolation of pure cultures of specific bacteria from a mixed sample.

Lyophilization may be performed by freezing the bacterial composition to a low temperature (e.g., below −40° C.), vacuuming the composition, sublimating the composition (i.e., changing the phase of ice in the sample directly from solid phase to vapor phase without becoming a liquid, desorption (i.e., removing remaining water molecules), and sealing to prevent water reentry. Protective reagents (e.g., sugars such as raffinose) may be included in the composition to help maintain survival. The lyophilized bacteria may be stored in a cool (e.g., 4° C. to 10° C.) and dark environment.

The sample obtained 130 in the process may be a water sample. In some embodiments, the water sample is a drinking water sample. The sample may be obtained from a potentially contaminated site.

The sample may be processed 140 prior to contacting the bacteria. Non-limiting examples of processing include filtering, concentrating, and/or removing iron.

Filtration may remove contaminants that would inhibit further processing and/or make fluorescence testing more challenging.

Non-limiting examples of concentration techniques include evaporation, ultrafiltration, and utilization of super-absorbent polymer (SAP) beads. In general, concentration may increase the concentration of PFAS in the sample, thereby allowing detection in the sample wherein the pre-processed sample had a PFAS concentration below a level that would typically be detectable in subsequent fluorescence observation.

In can be desirable to remove iron from the sample before processing, as the fluorophore that is induced is a siderophore (iron-chelating molecule).

Iron may be removed from the sample via oxidation. For example, the iron in the sample may be converted to an insoluble form and then filtered out.

In some embodiments, the iron is oxidized. Oxidation may follow the addition of a chemical agent or the injection of air or oxygen. Ferric iron formed via oxidation can be filtered out. More complex techniques are also contemplated, such as ion exchange and reverse osmosis. However, less expensive means may be preferable.

There is potential for this dose-dependent fluorescent response to other PFAS (e.g., perfluorohexanesulfonic acid) in Pseudomonas sp. CBR-F and other related bacterial isolates, and as such, could be used to detect those PFAS as well. This technique has potential to indicate PFAS levels in samples containing more than one PFAS compound (e.g., PFOS and PFOA). Another prospective application is detection of other halogenated compounds, like polychlorinated biphenyls (PCBs).

The bacteria and sample are mixed 150 to form a mixture. The bacteria may be added to a container holding the sample or the sample may be added to a container holding the bacteria. It is also possible that both the sample and the bacteria are added to a third container.

Mixing may be active or passive. In some embodiments, the mixture is stirred or agitated.

The mixture may be incubated 160. For example, the mixture may be incubated for a time period of from about 12 hours to about 16 hours.

In some embodiments, the incubation is at a temperature in a range of from about 25° C. to about 35° C.

Fluorescence may be detected or measured using a fluorometer. Generally, molecules occupy the lowest energy state (known as the ground state). Within this ground state are vibrational levels. Before becoming excited, many molecules occupy the lowest vibrational level.

An absorbed photon can cause a molecule to adopt a higher vibrational energy state when a molecule absorbs a certain wavelength of light. The molecules then collide with other molecules, causing a loss of vibrational energy and return to the lowest vibrational level of the excited state. The molecule can then return to ground state vibrational levels.

When the molecule returns to the ground state, it emits a photon of light at a wavelength different to the wavelength that excited it. When this takes place, the molecule exhibits fluorescence.

The fluorometer is an instrument designed to measure the various parameters of fluorescence, including intensity and wavelength distribution of the emission after excitation. Molecules capable of exhibiting fluorescence are commonly referred to as fluorophores.

Fluorescence spectrometers excite fluorophore molecules and measure emitted fluorescence. The spectrometer introduces UV or visible light using a photon source, (e.g., a laser, a xenon lamp, LEDs). Light passes through a monochromator that selects a specific wavelength, sometimes using a diffraction grating.

The spectrometer focuses the monochromatic wavelength towards the sample. The sample emits a wavelength, which travels to the detector which may be set at a 90-degree angle to the light source to avoid any interference from the transmitted excitation light. Emitted photons hit the detector and computer software connected to the detector can generate data and/or a graphical representation showing wavelengths that the sample absorbs. An emission spectrum shows which wavelengths the samples emit.

In connection with the present method, the fluorescence response is linear relative to the log of the PFOA/PFOS concentration, and likely other PFAS, thereby allowing PFAS concentration to be determined.

The following examples are provided to illustrate the devices and methods of the present disclosure. The examples are merely illustrative and are not intended to limit the disclosure to the materials, conditions, or process parameters set forth therein.

### EXAMPLES

To perform an assay, CBR-F was inoculated into medium containing Reasoner's 2A medium (R2A) diluted from a 2× stock with the test sample in a 96 well plate. Internal standards using PFOA are run each time as a calibration curve. Following incubation for a number of hours (for example, 12-16 hours at 30° C.), fluorescence was measured at 475 nm with an excitation of 425 nm after the cultures have grown with shaking at 150 rpm for 15 hours using a 96 well plate reader with fluorescent capabilities. The log of PFOA concentration is plotted against the measured fluorescence, and a linear trend is calculated. The trendline formula is used to calculate PFOA/PFOS concentrations in the sample.

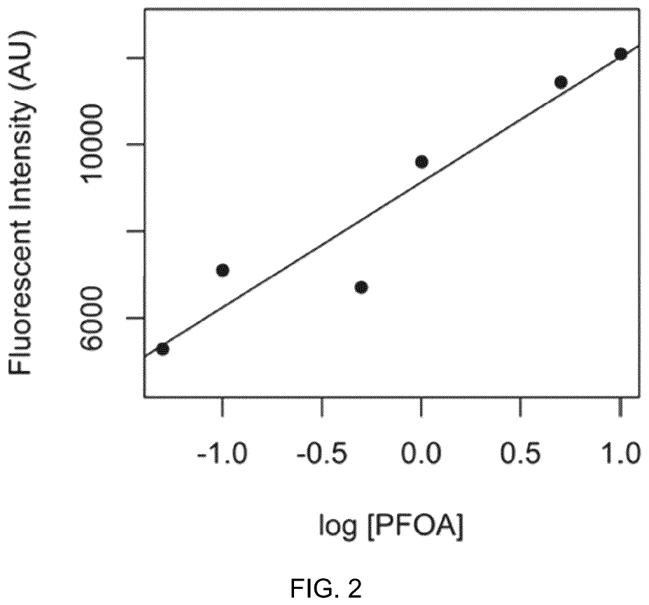

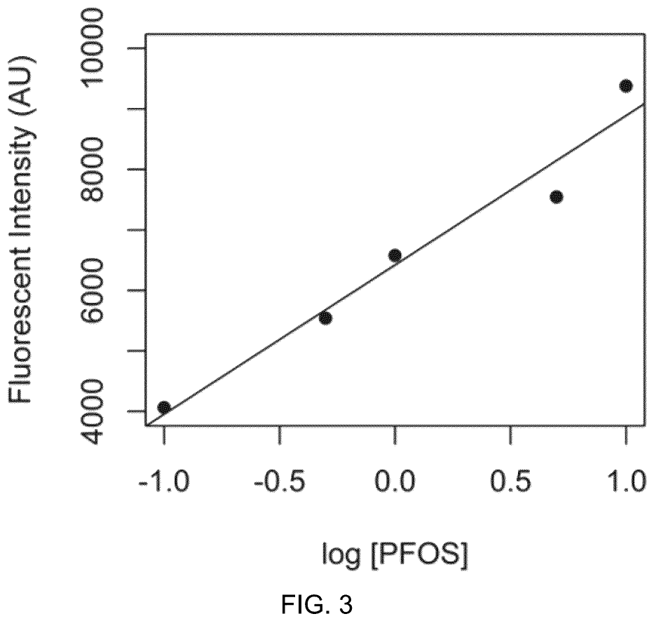

Data shows that this isolate Pseudomonas sp. CBR-F (deposited with the Agricultural Research Culture Collection on 19 Jul. 2023 with a deposit number of NRRL B-68295) produces fluorescence in response to both PFOA (FIG. 2) and PFOS (FIG. 3). The response is linear relative to the log of the PFOA/PFOS concentration, and has a limit of detection of 50 ppb (micrograms/liter).

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A method of detecting a perfluoroalkyl material, the method comprising:
contacting a strain of Pseudomonas sp. bacterium, the strain having been deposited with the Agricultural Research Culture Collection on 19 Jul. 2023 with a deposit number of NRRL B-68295, with a sample to form a mixture; and
observing the mixture to detect fluorescence thereof, wherein such fluorescence indicates the presence of the perfluoroalkyl material in the sample.

2. The method of claim 1, wherein the perfluoroalkyl material is perfluorooctanoic acid (PFOA).

3. The method of claim 1, wherein the perfluoroalkyl material is perfluorooctanesulfonic acid (PFOS).

4. The method of claim 1, further comprising:
culturing the strain prior to contacting the strain with the sample.

5. The method of claim 1, wherein the strain is provided in a liquid.

6. The method of claim 5, wherein the strain was lyophilized prior to being provided to the liquid.

7. The method of claim 1, wherein the method can detect the fluorine-containing material in the sample at a concentration of 50 micrograms per liter.

8. The method of claim 1, further comprising:
filtering the sample prior to the contacting.

9. The method of claim 1, further comprising:
concentrating the sample prior to the contacting.

10. The method of claim 1, further comprising:
removing iron from the sample prior to the contacting.

11. The method of claim 1, further comprising:
incubating the mixture for a predetermined time period prior to the observing.

12. The method of claim 11, wherein the predetermined time period is in a range of about 12 hours to about 16 hours.

13. The method of claim 11, wherein the incubation is at a temperature in a range of about 25° C. to about 35° C.

14. The method of claim 11, wherein the sample is agitated during the incubation.

15. The method of claim 1, wherein the sample is a water sample.

16. The method of claim 1, wherein the method is performed on a plurality of samples in parallel.

17. The method of claim 1, wherein observing the mixture comprises measuring fluorescence intensity.

18. The method of claim 1, further comprising:
determining a concentration of the perfluoroalkyl material in the sample based on the fluorescence intensity.

19. A lyophilized strain of Pseudomonas sp. bacterium, the strain having been deposited with the Agricultural Research Culture Collection on 19 Jul. 2023 with a deposit number of NRRL B-68295, wherein the strain, without having been modified, exhibits fluorescence when in the presence of perfluorooctanoic acid (PFOA) or perfluorooctanesulfonic acid (PFOS).

20. A composition comprising a lyophilized strain of Pseudomonas sp. bacterium, the strain having been deposited with the Agricultural Research Culture Collection on 19 Jul. 2023 with a deposit number of NRRL B-68295, wherein the strain, without having been modified, exhibits fluorescence when in the presence of perfluorooctanoic acid (PFOA) or perfluorooctanesulfonic acid (PFOS).
